(19) United States
(12) Patent Application Publication
(10) Pub. No.: US 2025/0177028 A1
(43) Pub. Date: Jun. 5, 2025

(54) SYSTEMS AND METHODS FOR IMPROVING CONTROL OF REFRIGERANT FLOW IN CRYOABLATION

(71) Applicant: Medtronic Cryocath LP, Toronto (CA)

(72) Inventors: Rachid Mahrouche, Cote St-Luc (CA); Eric Monger, Beloeil (CA); Wing-Choi Ma, Maple Grove, MN (US); Julia A. Schraut, Shoreview, MN (US); Bertin Simeon, Laval (CA); Timothy A. Ebeling, Circle Pines, MN (US)

(21) Appl. No.: 18/843,503

(22) PCT Filed: Mar. 30, 2023
(86) PCT No.: PCT/CA2023/050429

§ 371 Date: Sep. 3, 2024

Related U.S. Application Data
(60) Provisional application No. 63/328,408, filed on Apr. 7, 2022.

(51) Int. Cl. A61B 18/02; A61B 18/00

(52) U.S. Cl. CPC A61B 18/0218; A61B 2018/0022; A61B 2018/00577; A61B 2018/00648; A61B 2018/00773; A61B 2018/00791; A61B 2018/0212; A61B 2018/0268

(57) ABSTRACT

Systems and methods for cryoablation. One example system includes an interior portion, an exterior portion and a treatment element. The system includes a first loop in the interior portion of the system, the first loop having a first controller, at least one valve, and a first sensor; a second loop in the interior portion of the system, the second loop having a second controller, and at least one valve, and a second sensor; a pressurized coolant supply in fluid communication with the first loop and the second loop; and a temperature sensor disposed on the exterior portion of the system, the temperature sensor being configured to monitor the temperature on the exterior portion of the system.

### CROSS-REFERENCE TO RELATED APPLICATION

The present application is related to and claims benefit under 35 U.S.C. § 119(e) from U.S. Provisional Patent Application Ser. No. 63/328,408, filed 7 Apr. 2022, entitled “METHOD FOR IMPROVING CONTROL OF REFRIGERANT FLOW IN CRYOABLATION”, the entire contents of which being incorporated herein by reference.

### FIELD

The present technology is generally related to a method and system for controlling the flow of liquids and gasses in medical devices.

### BACKGROUND

Cryoablation is a type of procedure used in electrophysiology for treatment of various cardiac issues including cardiac arrhythmias such as atrial fibrillation. During cryoablation, a catheter may be used percutaneously to create lesions on tissue within the heart. These lesions may be created by the rapid removal of heat from the cardiac cells. This energy transfer is made possible by the use of a cryoablation catheter, a cryoablation console and a refrigerant source. The cryoablation console may be used to deliver refrigerant to the catheter (e.g., injected into a treatment element within the catheter). The refrigerant may be injected into the treatment element in a contracted state and once it comes into contact with the warmth of tissue, the refrigerant may expand and this expansion of the refrigerant may trigger the recovery of the expanded refrigerant by the console.

Generally, consoles control the flow of refrigerant going into the catheter using one or more proportional integral derivative (“PID”) feedback control algorithms. For example, refrigerant that is injected into the catheter may be controlled by measuring a return path flow rate to determine how much to open an injection proportional valve as well as by using temperature control to measure the temperature at the treatment element and use this measured value to determine how much to open the injection proportional valve.

The console may also have a vacuum line with a one or more PID feedback control algorithms to control a vacuum proportional valve which is often used to ensure that inflatable treatment elements maintain a specific internal pressure.

During cryoablation, it is important to maintain precise temperature control which is when the treatment device is a focal catheter for mapping. Mapping during cryoablation temporarily stuns certain tissue rather than creating a permanent lesion and is used to locate arrhythmogenic sources within, for example, the heart. During mapping, the treatment device's temperature is generally controlled and may be as low as −30 degrees Celsius and above to stimulate tissue and cause a change in the signal. Due to the delicate nature of this type of mapping, the temperature control requires low flow rates of refrigerant as well as precise temperature control. When low flow rates are required, it can often be difficult to control the temperature which can result in overshooting and undershooting temperatures.

Also, it can be difficult with PID feedback control algorithms to control low flow rates of refrigerant using a single size injection proportional valve when high pressure refrigerant is being used. The injection proportional valve must have large enough openings to allow high flow rates for large catheters as well as flow across a large range of inlet and outlet pressures.

### SUMMARY

The techniques of this disclosure generally relate to systems, methods, and various apparatuses for use with a medical device.

In one aspect, there is a system for cryoablation, the system may have a first pathway and a treatment element. The first pathway may be in communication with the treatment element and a first controller, at least one first valve, and a sensor, the first controller, the at least one first valve, and the sensor may all be disposed in the first pathway. The system may also include a second controller and a second valve, the second controller and the second valve may be in communication with the first pathway, and a pressurized coolant supply may be in fluid communication with the first pathway.

According to this aspect, the first controller is a first proportional integral derivative controller and the second controller is a second proportional integral derivative controller.

According to this aspect, the system may further comprise an interior portion and an exterior portion, the exterior portion of the system including a temperature sensor configured to record the temperature on the exterior portion of the system and the temperature sensor is in communication with the first controller.

According to this aspect, the exterior portion of the system may further include a humidity sensor configured to record the humidity on the exterior portion of the system and the humidity sensor is in communication with the first controller.

According to this aspect, the first pathway is configured to be activated when the treatment element is in a mapping mode and when the treatment element is in the mapping mode, the first controller sets a mapping pressure based upon the temperature measured by the temperature sensor at the exterior portion.

According to this aspect, at least one first valve has an inlet and an outlet, the mapping pressure is determined based upon the inlet pressure from the at least one first valve which is measured by the first sensor.

According to this aspect, the first controller is configured to proportionally decrease the mapping pressure based upon a first preset temperature range that is measured by the temperature sensor.

According to this aspect, wherein the second controller and the second valve are disposed within the first pathway.

According to this aspect, the system may further comprise a second pathway, the second pathway being a vacuum pathway and the first pathway being an injection pathway.

According to this aspect, the second controller and the second valve are disposed within the second pathway.

In another aspect, the system for cryoablation may have an interior portion, an exterior portion and a treatment element. The system may comprise a first pathway in the interior portion of the system and the first pathway may have a first proportional integral derivative controller, a first valve, and a first pressure transducer. The system may also have a second pathway in the interior portion of the system and the second pathway may be in communication with a scavenging connector. The second pathway may also have a second proportional integral derivative controller and a second valve. A pressurized coolant supply may be in fluid communication with the first pathway. A connector may be in communication with the treatment element, the connector having a vacuum lumen and an injection lumen.

According to this aspect, the vacuum lumen and the injection lumen are in fluid communication with the treatment element through the connector.

According to this aspect, the system may further comprise a temperature sensor, the temperature sensor may be disposed on the exterior portion of the system and be configured to record the temperature on the exterior portion of the system.

According to this aspect, the first pathway may be configured to be activated when the treatment element is in a mapping mode and when the treatment element is in the mapping mode the first proportional integral derivative controller may set a mapping pressure based upon the temperature measured by the temperature sensor.

According to this aspect, the first valve has an inlet and an outlet, the mapping pressure is determined based upon an inlet pressure from the at least one first valve which is measured by the first pressure transducer.

According to this aspect, the first proportional integral derivative controller is configured to proportionally decrease the mapping pressure based upon a first preset temperature range that is measured by the temperature sensor.

In another aspect, a method for mapping with a treatment element where the treatment element is part of a system for cryoablation may include a console and an umbilical system coupling the console to the treatment element. The method may comprise the steps of setting the console in a mapping mode and setting a mapping pressure based on a temperature as measured on an exterior portion of the console.

According to this aspect, the mapping pressure is configured to decrease proportionally as the temperature measured on the exterior portion of the console decreases.

According to this aspect, the mapping pressure is configured to increase proportionally as the temperature measured on the exterior portion of the console increases.

According to this aspect, the mapping pressure is configured to decrease proportionally as the humidity measured on the exterior portion of the console increases.

According to this aspect, the mapping pressure is configured to increase proportionally as the humidity measured on the exterior portion of the console decreases.

In another aspect, a system for cryoablation, where the system may have an interior portion, an exterior portion and a treatment element. The system may comprise a first pathway in the interior portion of the system. The first pathway may have a first controller and a first valve and a second controller and a second valve disposed proximate the first controller. A pressurized coolant supply may be in fluid communication with the first pathway.

According to this aspect, the first valve is an injection proportional valve and the first controller is a proportional integral derivative controller.

According to this aspect, the second valve is a vent proportional valve and the second controller is a proportional integral derivative controller.

In another aspect, a system for cryoablation may have an interior portion, an exterior portion and a treatment element. The system may comprise a first pathway in the interior portion of the system. The first pathway may have a first controller, a first valve, a second valve, and a first sensor. A second pathway may be in the interior portion of the system, the second pathway may be in communication with the first pathway and the second pathway may have a second controller and a third valve. A connector may be in communication with the first pathway, the second pathway and the treatment element and a pressurized coolant supply in fluid communication with the first pathway and the second pathway.

According to this aspect, the connector is a coaxial connector with a first umbilical lumen and a vacuum lumen.

According to this aspect, the first valve is an injection proportional valve and the first controller is a proportional integral derivative controller.

According to this aspect, the third valve is an injection proportional valve.

According to this aspect, the second controller is a proportional integral derivative controller.

According to this aspect, the second controller is in communication with the first pathway and the first controller.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF THE DRAWINGS

A more complete understanding of the present invention, and the attendant advantages and features thereof, will be more readily understood by reference to the following detailed description when considered in conjunction with the accompanying drawings wherein:

FIG. 1 illustrates an example treatment device that may be used in conjunction with the present invention;

FIG. 2 is a schematic representing the mechanical components of the control console of the present invention;

FIG. 3 is a schematic representing the mechanical components of the control console of the present invention;

FIG. 4 is a simplified schematic representing various components of the control console of the present invention; and

FIG. 5 is a graph showing the refrigerant pressure and the temperature when the control console is in use.

### DETAILED DESCRIPTION

Before describing in detail example embodiments, it is noted that the embodiments reside primarily in combinations of apparatus components and processing steps related to feedback control systems for cryo-mapping and cryoablation. Accordingly, components have been represented where appropriate by conventional symbols in the drawings, showing only those specific details that are pertinent to understanding the embodiments so as not to obscure the disclosure with details that will be readily apparent to those of ordinary skill in the art having the benefit of the description herein. Like numbers refer to like elements throughout the description.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors, application specific integrated circuits (ASICs), field programmable gate arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

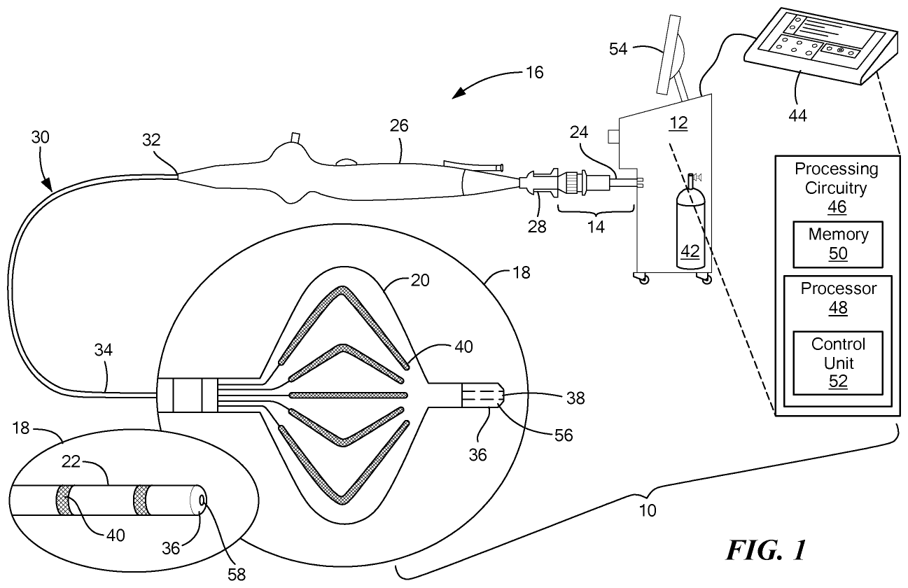

Referring now to the drawing figures where like elements have like reference numerals, FIG. 1 illustrates an example catheter system 10 that may be used to map and treat tissue. The catheter system 10 may include a console 12 coupled to one end of an umbilical system 14. The opposing end of the umbilical system 14 may be coupled with a medical device 16. The medical device 16 may be any medical device including a medical device with an inflatable portion including a catheter and a balloon catheter or it may be a medical device with a focal catheter. As shown in FIG. 1, the medical device 16 may include a treatment portion 18 and as shown in FIG. 1 and the treatment portion 18 may include an inflatable portion 20 and/or a focal catheter 22.

The umbilical system 14 may include a coaxial cable umbilical 24 which includes both a cooling injection umbilical and a vacuum umbilical that provide respective inlet and return paths for refrigerant or coolant to cool a tissue-treating end of the treatment portion 18 of the medical device. The vacuum umbilical may be used to allow excess coolant or gas to escape from the medical device 16 and the cooling injection umbilical may transfer coolant from the console 12 to the medical device 16 to be used for various treatments of tissue.

Continuing to refer to FIG. 1, the console 12 may be coupled to the medical device 16 via the umbilical system 14 and the medical device 16 may include a handle 26 having a coaxial connector 28 with both an injection lumen and vacuum lumen therein. The medical device 16 may further include an elongate body 30 having a proximal portion 32 and a distal portion 34 which define a fluid flow path and a distal end 36, the distal end 36 may include a lumen 38 therein. The treatment portion 18 of the medical device 16 may be coupled to the elongate body 30. The elongate body 30 may be a flexible tubing that couples fluid from a fluid source to the treatment portion 18 and the distal portion 34 of the elongate body 30. For example, cold fluid including refrigerant and/or coolant may be injected into the cooling injection umbilical within the elongate body 30 to the treatment portion 18 of the medical device 16. The vacuum umbilical within the elongate body 30 may evacuate the injected fluid from the treatment portion 18 back to the console 12.

The treatment portion 18 may have at least one electrode 40 to contact surrounding tissue and sense electrical activity in the tissue in the vicinity of the at least one electrode 40. It will be understood that the at least one electrode 40 may be one or more than one electrode in a variety of different shapes, sizes, and configurations including band and ring electrodes. The catheter system 10 may be generally include the medical device 16 that may be coupled directly to a coolant supply and/or an energy supply configured to deliver coolant to the medical device 16 and/or generate and deliver various modalities of energy. Although not shown, the console 12 may include an energy supply source that is in communication with the medical device 16 and the treatment portion 18 of the medical device 16. For example, the console 12 may be coupled with a coolant source 42 configured to deliver the coolant source 42 to the medical device 16. It will be understood that the reference to coolant source 42 will also include a refrigerant source as well.

A remote controller 44 may be in communication with the console 12 and the coolant source 42, the remote controller 44 including processing circuitry 46 configured to operate and control the various functions of the console 12 and the coolant source 42 and in further communication with the inflatable portion 20 of the medical device 16 or the focal catheter 22 of the medical device 16. Alternatively, the remote controller 44 may be integrated within the console 12. The medical device 16 may generally include one or more diagnostic or treatment regions for energetic, therapeutic and/or investigatory interaction between the medical device 16 and a treatment site. The treatment region(s) may deliver, for example, a coolant supply to the inflatable portion 20 of the medical device 16 sufficient to cryoablate a tissue area.

In one or more embodiments, the processing circuitry 46 may include a processor 48 and a memory 50. In particular, in addition to or instead of a processor 48, such as a central processing unit, and memory, the processing circuitry 46 may comprise integrated circuitry for processing and/or control, e.g., one or more processors and/or processor cores and/or FPGAs (Field Programmable Gate Array) and/or ASICs (Application Specific Integrated Circuitry) adapted to execute instructions. The processor 48 may be configured to access (e.g., write to and/or read from) the memory 50, which may comprise any kind of volatile and/or nonvolatile memory, e.g., cache and/or buffer memory and/or RAM (Random Access Memory) and/or ROM (Read-Only Memory) and/or optical memory and/or EPROM (Erasable Programmable Read-Only Memory).

The processing circuitry 46 may be configured to control any of the methods and/or processes described herein and/or to cause such methods, and/or processes to be performed, e.g., by the remote controller 44. Processor 48 corresponds to one or more processors 48 for performing functions described herein. The memory 50 is configured to store data, programmatic software code and/or other information described herein. In some embodiments, the software may include instructions that, when executed by the processor 48 and/or processing circuitry 46 causes the processor 48 and/or processing circuitry 46 to perform the processes described herein with respect to remote controller 44. For example, processing circuitry 46 of the remote controller 44 may include a control unit 52 that is configured to perform one or more functions described herein.

When fluid is injected into the treatment portion 18 via the elongate body 30, the treatment portion 18 may expand, causing the electrodes 40 to contact surrounding tissue and sense electrical activity in the tissue in the vicinity of the electrodes 40. The electrical activity sensed by the electrodes 40 may be conducted by wires to equipment that records and displays the electrical activity. For example, the console 12 may have a display 54 which displays the electrical activity of the electrodes 40 as well as various other components of the medical device 16. Additionally, the treatment portion 18 may be the focal catheter 22 and the focal catheter 22 may be placed in contract with or in close proximity to tissue and the electrodes 40 may sense electrical activity in the tissue in the vicinity of the electrodes 40. Such equipment may include an electrocardiograph (ECG), a computer with a keyboard, mouse, and video monitor as shown as the display 54. The signals that are displayed on the display 54 may assist the medical provider in determining, among other things, an amount of electrical activity of the tissue and how well the treatment portion 18 is making contact with the surrounding tissue and whether the ablation and/or cryoablation in a current position is likely to succeed or whether the treatment portion 18 should be repositioned and a new mapping obtained.

In addition to, or in the alternative to the treatment portion 18, the distal end 36 of the elongate body 30 may be equipped with a thermal tip 56 that removes heat from the tissue to reach a first temperature for cryo-mapping and to reach a second temperature that ablates the tissue. The target temperature for the thermal tip 56 is higher for cryo-mapping than for cryoablation, so that for cryo-mapping the cellular effects are reversible and for cryoablation the cellular effects are irreversible.

Coolant may be provided by the coolant source 42 within the console 12. The coolant may be N₂O or another type of cooling gas and/or liquid may pass through the internal piping of the console 12 before being transferred to the medical device 16 via the coaxial cable umbilical 24. At the distal end of the umbilical, inside the medical device 16, the coolant may be released inside the catheter tip cavity, which is under vacuum. Both the phase change from liquid to gas and the sudden expansion of the coolant are endothermic reactions, causing a temperature differential which may result in the catheter tip or balloon freezing. The coolant vapor may then be returned through the vacuum path of the umbilical system 14 and into the console 12, where it may be evacuated. The delivery of refrigerant into the medical device 16 may be controlled by measuring the temperature, for example, with a sensor 58 near the distal end 36 of the medical device 16. It will be understood that the sensor 58 may be any type of sensor including a temperature and/or pressure sensor.

As described herein, console 12 may include a first pathway and a treatment element (e.g., medical device 16), the first pathway being in communication with the treatment element. Console 12 may also include a first controller 44, which may be included in the processing circuitry 46, at least one first valve, and a sensor, the first controller, the at least one first valve, and the sensor all being disposed in the first pathway. Console 12 also includes a second controller (e.g., included in processing circuitry 46) and a second valve, the second controller and the second valve being in communication with the first pathway. Coolant source 42 provides a pressurized coolant supply in fluid communication with the first pathway. By including the first pathway and the second pathway that are in communication, console 12 allows for more precise temperature control of the coolant as well as greater control of the flow rate of the coolant within the console 12 and the medical device 16.

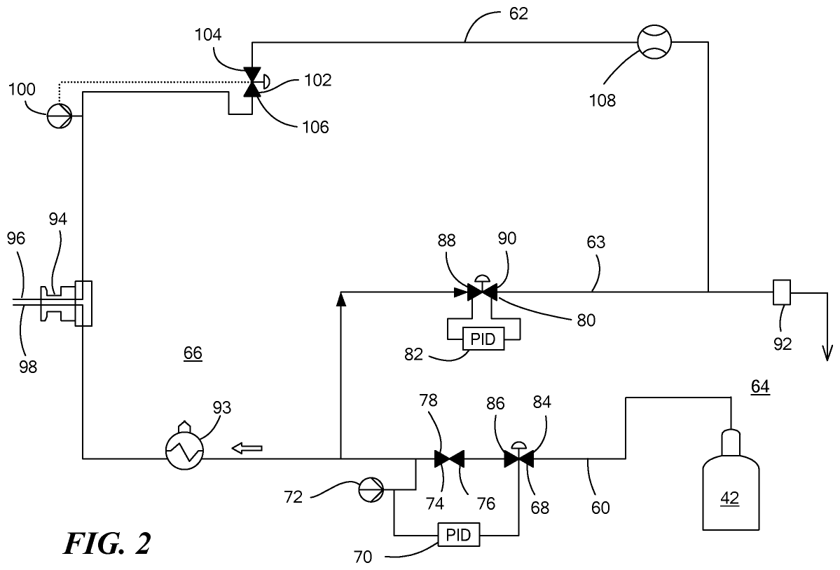

Now referring to FIG. 2, a schematic representation of an example system within the console 12 portrayed in FIG. 1 for the delivery and removal of coolant and/or refrigerant to the medical device 16. The schematic shown is designed for use with a medical device 16 including the focal catheter 22 and the treatment portion 18 with the inflatable portion 20, with the example system showing a layout of example internal mechanical components of the console 12.

The console 12 may include a first pathway 60 and a second pathway 62, as shown in FIG. 2. Additionally, the console 12 may include a third pathway 63. The first pathway 60 may include different components in different configurations and the first pathway 60 may be a high-pressure pathway that makes fluid flow from the coolant source 42 into the medical device 16. The first pathway 60 may generally include a tank area 64 and a heat exchanger area 66. The second pathway 62 may be a low-pressure pathway to remove fluids and gasses from the medical device 16.

Continuing to refer to FIG. 2, the first pathway 60 may include a first valve 68 and the first valve 68 may be connected to a first controller 70 and the first controller 70 may be a first proportional integral derivative feedback controller (“PID”) and this first controller 70 may be programmed to cause certain components of the console 12 to open and close. In general, a PID controller may be used to regulate temperature, flow, pressure, speed, and other process variables. The PID controller operates by using a closed-loop feedback mechanism to control process variables and to keep the actual output from a process as close to the target or setpoint output as possible. There are also various types of non-PID controllers which offer alternative solutions. For example, a thermostatic controller could be used to maintain a constant temperature by, for example, turning the heater on when the temperature drops below a certain setpoint or turning the heater off when the temperature rises too high. Also, a multivariable controller may be used when the controller is coordinating the efforts of multiple actuators to control multiple process variables simultaneously.

The first controller 70 may be in communication with a first sensor 72 and this first sensor 72 may be any type of sensor. For example, the first sensor 72 may be a pressure sensor which is configured to detect the amount of pressure at the inlet and/or outlet of the first valve 68. The first pathway 60 may also include a second valve 74 and the second valve 74 may have an inlet 76 and an outlet 78 that may be disposed opposite the inlet 76. In one embodiment, the second valve 74 may be a solenoid valve and this valve may be used for safety purposes as a means to stop the flow of coolant/refrigerant in the first pathway 60 if the coolant/refrigerant is not needed in the medical device 16. The second valve 74 may be a redundant valve of the first valve 68 to provide additional control over any coolant/refrigerant in the first pathway 60. It will be understood that each of the valves shown in the console 12 may have an inlet and an outlet and the inlet may be opened, the outlet may be opened, the inlet may be closed, the outlet may be closed, and or any combination of opening and closing of the inlets and outlets may be used depending upon how gas or liquid is to flow within the console 12.

As shown in FIG. 2, the first sensor 72 may be a pressure sensor and the pressure measurements from the first sensor 72 that are detected by the first controller 70 may cause the opening and closing of the first valve 68 and/or the second valve 74 based upon the preset programming of the first controller 70. The coolant source 42 may be a source of nitrous oxide or any other type of coolant that may be used to inflate the medical device 16.

If too much refrigerant is introduced into the first pathway 60 the first valve 68 and the second valve 74 may be opened or closed to limit the amount of refrigerant that can pass through the first valve 68 to the second valve 74 in the first pathway 60. The first valve 68 and the second valve 74 may prevent the refrigerant from reaching the heat exchanger 66 area within the first pathway 60.

Also, the first pathway 60 may be in communication with a third valve 80 and a second controller 82 and the third valve 80 and the second controller 82 may be disposed in the third pathway 63 to help control the flow of fluid or gas within the first pathway 60. For example, the first valve 68 and the first controller 70 may control low and high pressure flow rates in the first pathway 60 and the flow rates which are desired can be variable depending upon the type of treatment being performed with the medical device 16. The first valve 68 may have enough capacity to be able to control the high and low flow rates and have an inlet 84 and an outlet 86 that are each sized to allow for high flow rates with larger medical devices, such as catheters, as well as lower flow rates with smaller medical devices. Further, the flow rates desired within the first pathway 60 may also depend upon the type of procedure being performed and the individual that is being treated.

When the first valve 68 is being used to control flow rates, the first valve 68 may sometimes allow too much gas or liquid to pass beyond the first valve 68 in the first pathway 60 from the coolant source 42 toward the medical device 16 and when this occurs the placement of the third valve 80 and the second controller 82 downstream from the first valve 68 can allow the third valve 80 and the second controller 82 in the third pathway 63 to precisely and quickly remove refrigerant from the first pathway 60 in the event that too much liquid or gas passes downstream from the first valve 68. The third valve 80 may have an inlet 88 and an outlet 90 and the third valve 80 may be connected to and/or in communication with the second controller 82 and the second controller 82 may be a PID controller and this second controller 82 may cause certain components of the console to open and close depending upon certain preset parameters. For example, the second controller 82 may be configured to open the third valve 80 to move coolant and refrigerant towards a scavenging connector 92 in the third pathway 63 of the console 12 to remove any excess refrigerant or coolant from the first pathway 60.

The second controller 82 may also be in communication with the first sensor 72 and this first sensor 72 may be any type of sensor. For example, the first sensor 72 may be a pressure sensor which is configured to detect the amount of pressure at the outlet 86 of the first valve 68. When certain pressure settings are detected by the first sensor 72, the second controller 82 may cause the opening and closing of the outlet 90 and the inlet 88 of the third valve 80 to assist with the removal of gas or liquid within the first pathway 60 to the third pathway 63 towards the scavenging connector 92. For example, the first valve 68 may have the outlet 86 and the inlet 84 closed and the first sensor 72 may detect a pressure that is higher than the preset target pressure set in either the first controller 70 and/or the second controller 82. When this occurs, the outlet 90 and/or the inlet 88 of the third valve 80 can be opened to assist with the removal of excess gas or liquid from the first pathway 60 and move the excess gas or liquid toward the third pathway 63 and ultimately toward the scavenging connector 92. Furthermore, depending upon the pressure measurements from the first sensor 72, the third valve 80 may be closed when the first valve 68 is opened and this may be controlled by the first controller 70 based upon the preset pressure measurements.

In the embodiment as shown in FIG. 2, the second controller 82 and the third valve 80 are located downstream from the first valve 68 and the second valve 74. However, the second controller 82 and the third valve 80 may also be located in different locations and/or within the second pathway 62 as well. Also, the second valve 74 may be absent from the console 12. Using the third valve 80 and the second controller 82 along with specific feedback control algorithms that are programmed into the console 12 can control the opening and closing of the third valve 80 and the opening and closing of the third valve 80 can help control the temperature of the liquid or gas within the treatment portion 18 of the medical device 16. When the temperature of the liquid or gas within the first pathway 60 is too high or too low, the second controller 82 can be used to control the opening and closing of the third valve 80 so that liquid or gas within the first pathway 60 may be evacuated toward the scavenging connector 92 to help regulate the temperature of the liquid and/or gas.

In this configuration, the coolant/refrigerant may be removed by the second controller 82 and the third valve 80 while the coolant/refrigerant is in the first pathway 60. The third valve 80 and the second controller 82 can be used to help control low flow rates within the first pathway 60 where, for example, the first valve 68 allows too much refrigerant or coolant to enter the first pathway 60. The second controller 82 can open the third valve 80 to remove fluid or gas from the first pathway 60 toward the scavenging connector 92. Use of the controllers can be used to more precisely control the flow of liquid or gas within the console which allows better flow rate control such that medical devices, including low-flow rate catheters, adjustable balloon catheters, and compliant balloon catheters can all be used with console 12 while maintaining reliable and controllable flow rates. Additionally, downstream from the first valve 68 and the second valve 74 in the first pathway 60, may be a heat exchanger 93. When the liquid and/or gas is in the first pathway 60 moving from the coolant source 42 toward a connector 94, the liquid and/or gas may pass through the heat exchanger 93 which may be used to transfer heat from one process stream to another.

Continuing to refer to FIG. 2, the connector 94 may be any type of connector 94 that may connect the medical device 16 to the console 12. In one embodiment, the connector 94 may be a coaxial connector 94 having both a vacuum lumen 96 and an injection lumen 98. As part of the vacuum system in the console 12, the second pathway 62 may be a low-pressure pathway that is in communication with the scavenging connector 92. The second pathway 62 may be a low-pressure pathway and may include a variety of different components and configurations to enable liquid or gas to be retrieved from the medical device 16 back into the console 12 toward the scavenging connector 92. When gas and/or liquid enters the second pathway 62, the gas or liquid may move toward a second sensor 100 and the second sensor 100 may be in communication with a fourth valve 102 and the measurements made by the second sensor 100 may cause the opening and closing of an outlet 104 and an inlet 106 of the fourth valve 102. The measurements made by the second sensor 100 may be used to maintain a particular preset pressure measurement within the medical device 16 by opening and/or closing the fourth valve 102 in the second pathway 62.

Downstream from the fourth valve 102 in the second pathway 62 may be a first meter 108 and the first meter 108 may be any type of meter including a flow meter to measure the mass or the flow rate of fluid or gas passing through the first meter 108. The scavenging connector 92 may connect to an external scavenging collection unit (not shown), allowing gas and/or liquid refrigerant from within the console 12 to be disposed of from the external scavenging collection unit. The flow of refrigerant within the console 12 may be controlled in a number of other ways as well. For example, the flow rate in the second pathway 62 may be measured, for example by one of the sensors such as by the first meter 108, and the flow rate measured by the first meter 108 may be communicated to the first controller 70 and the first controller 70 can control the opening and closing of the first valve 68 as well as how much to open and/or close the inlet 84 and/or the outlet 86 of the first valve 68.

Furthermore, the delivery of refrigerant into the medical device 16 may be controlled by measuring, for example, the temperature of the refrigerant that is proximate the sensor 58 in the treatment portion 18 of the medical device 16. The temperature measurement may by the sensor 58 may be communicated to the first controller 70 and/or the second controller 82 and this measurement can be used to control the opening and/or closing of the first valve 68, the second valve 74, the third valve 80, and/or the fourth valve 102. The sensor 58 in the medical device 16 may be any type of sensor including a temperature and/or pressure sensor. For example, the value of the temperature measurement may control the opening and closing of the first valve 68 as well as how much to open and/or close the inlet 84 and the outlet 86 of the first valve 68. While the first controller 70 receives information, which dictates whether to open and/or close the inlet 84 and/or the outlet 86 of the first valve 68, the second pathway 62 which may be a vacuum pathway, may in an alternative embodiment, have a controller that is configured to receive pressure measurements from the sensor 58 in the medical device 16 to help the inflatable portion 20 maintains a specific preset pressure by allowing for the controlled opening and closing of the first pathway 60 and the second pathway 62.

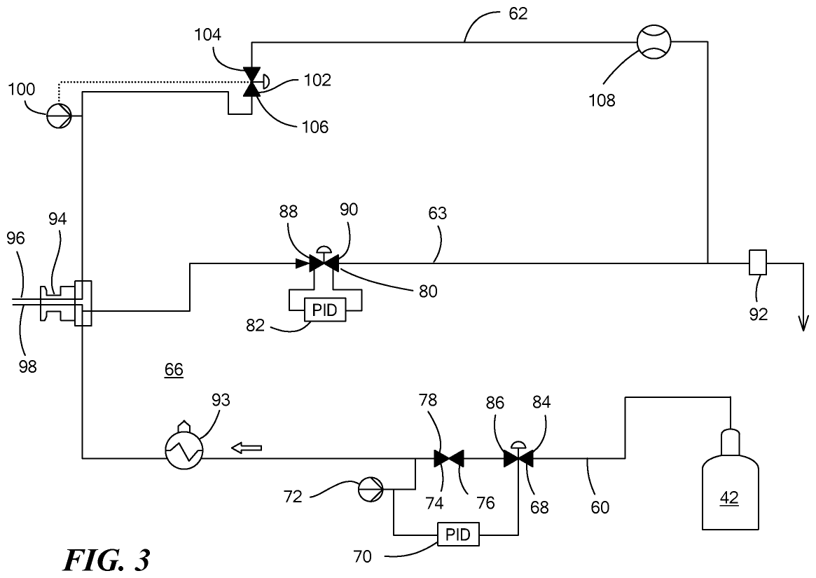

Now referring to FIG. 3 is another schematic of the console 12 where the second controller 82 and the corresponding third valve 80 are disposed in a different location than what is shown in FIG. 2. It will be understood that when describing the components of the console in FIGS. 2-3, that the components may be arranged in different ways and that the console 12 may not include all of the components as shown in the example embodiments or the console 12 may include additional components which are not shown. In this configuration, the various components within the console 12 may generally be the same as those shown in FIG. 2, but the location of the second controller 82 and the third valve 80 may be different than the location of these components in the schematic shown in FIG. 2. Having the second controller 82 and the third valve 80 in the third pathway 63 and the third pathway being in communication with the connector 94 may allow any excess refrigerant that is introduced by the first valve 68 in the first pathway 60 to enter into the heat exchanger 93. Once the excess refrigerant is in the heat exchanger 93, depending upon the preset settings of the second controller 82 and/or the first controller 70, excess refrigerant that passes through the heat exchanger 93 toward the connector 94 may be removed from the first pathway 60 to the third pathway 63 by opening the inlet 88 or outlet 90 of the third valve 80 to move the excess refrigerant toward the scavenging connector 92 in the third pathway 63.

As shown in FIGS. 2 and 3, the opening and closing of the third valve 80 in the third pathway 63 may be controlled by the preset settings of the second controller 82 to move coolant/refrigerant from the first pathway 60 into the third pathway 63 toward the scavenging connector 92. Where the second controller 82 and the third valve 80 are in communication with the first pathway 60 can allow for the quick and precise removal of coolant/refrigerant from the first pathway 60 into the third pathway 63 before the coolant/refrigerant enters the medical device 16. The placement of these components can prevent any excess refrigerant that may have been introduced into the first pathway 60 from going into the medical device 16. The first valve 68 may also be in communication with the third valve 80 through the first controller 70 and/or the second controller 82 and this communication may control the opening and closing of the first valve 68, the second valve 74, and/or the third valve 80 to control the movement and flow of liquid and/or gas through the console 12.

A PID control algorithm may be used by the second controller 82 to control the opening and closing of the third valve 80. The settings of the algorithm can determine when the second controller 82 activates the opening and closing of the outlet 90 and the inlet 88 of the third valve 80. A PID algorithm may be used when the actual pressure detected within the first pathway 60 does not match a particular preset target pressure. For example, if the pressure detected within the first pathway 60 is higher than the preset target pressure, the PID algorithm may cause the second controller 82 to open the inlet 88 and the outlet 90 of the third valve 80 so that refrigerant/coolant may be removed from the first pathway 60 into the third pathway 63. Optionally, when the inlet 84 and outlet 86 of the first valve 68 are closed and the first sensor 72 is measuring a pressure that is higher than the preset target pressure in the first pathway 60, the third valve 80 can be opened to remove any excess gas or liquid within the first pathway 60 into the third pathway 63. Furthermore, the third valve 80 can be closed at a certain preset pressure parameter or temperature parameter and the first valve 68 may be opened based upon the preset parameters of the first controller 70 to maintain a particular pressure and/or temperature within the first pathway 60.

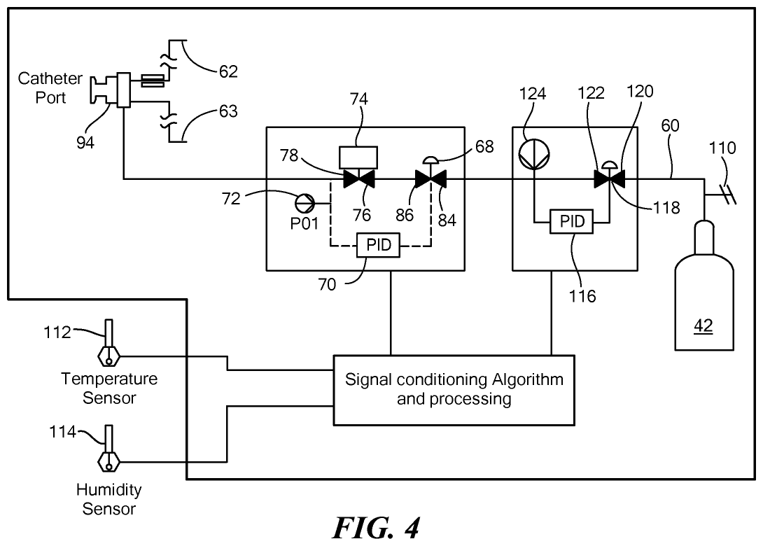

Now referring to FIG. 4 is another simplified schematic of the console 12 with additional components that can be used to control and maintain the pressure within the first pathway 60. In the first pathway, the coolant source 42 may be in communication with a fifth valve 110 that may be located upstream from the coolant source 42. The fifth valve 110 may be a check valve to ensure that the flow of any refrigerant/coolant only goes from the coolant source 42 down the first pathway 60 toward the connector 94 and that the coolant/refrigerant from the coolant source 42 does not run back toward the coolant source 42. The console 12 may include a third sensor 112, a fourth sensor 114, a third controller 116, a sixth valve 118 with an inlet 120 and an outlet 122, and a fifth sensor 124. The fifth sensor 124, the third controller 116, and the sixth valve 118 may be located in the first pathway 60, which may be the injection pathway. As shown in FIG. 4, the first pathway 60 may include the first controller 70 along with the first valve 68, the second valve 74 and the first sensor 72 and closer to the coolant source 42 in the same pathway may be a third controller 116 along with a sixth valve and a fifth sensor 124.

Continuing to refer to FIG. 4, the third controller 116 and the sixth valve 118 may be used to control the inlet 84 pressure of the first valve 68. The third controller 116 may be any type of controller, but as shown in FIG. 4, this controller may be a PID controller, the sixth valve 118 may be a proportional valve, and the fifth sensor 124 may be a pressure sensor. Also, the third sensor 112 and the fourth sensor 114 may be any type of sensor, including a temperature, humidity and/or pressure sensor which are located on the exterior of the console 12 to measure environmental conditions outside of the console 12 which may impact how the catheter system 10 works. For example, the third sensor 112 may be a temperature sensor configured to measure the environmental operating temperature of the console 12 outside of the console 12 and the fourth sensor 114 may be a humidity sensor configured to measure the environmental operating humidity outside the console 12. The environmental operating humidity of the console 12 may be the humidity level of the environment where the medical device 16 is being operated such as within a procedure room. The third sensor 112 may measure the environmental operating temperature outside of the console 12 and depending upon the temperature that is measured by the third sensor 112, at least one of the first controller 70 and/or the third controller 116 may open or close the inlet 84 of the first valve 68 to lower or raised the pressure based upon the recorded environmental operating temperature.

During cryo-mapping, the environmental operating temperature outside the console 12 may impact the pressure settings within the first, second, and/or third pathways 60, 62, and 63. The fourth sensor 114, which may be a humidity sensor, may be configured to determine the level of the humidity outside of the console 12 and the level of the humidity outside of the console 12 may also be used to adjust the pressure settings within the first, second, and/or third pathways 60, 62, and 63. In one example embodiment, the pressure setpoint in the first controller 70 may be changed based upon the determination of the humidity level by the fourth sensor 114 while no other pressure settings within the console 12 are adjusted based upon the humidity level measured by the fourth sensor 114.

Additionally, the pressure of the liquid and/or gas that is expelled from the coolant source 42 can vary about 300 psig depending upon the environmental operating temperature which is the temperature of the area outside of the console 12. For example, as the temperature measured by the third sensor 112 deceases, the setting of the mapping pressure within the console 12 may decrease. Alternatively, if the temperature measured by the third sensor 112 increases, the setting of the mapping pressure within the console 12 may increase. The third controller 116, the sixth valve 118, and the fifth sensor 124 may be used to control and adjust the inlet and outlet pressure of the first valve 68 while the console 12 is in use and treatment is being delivered using the medical device 16. The third controller 116, the sixth valve 118, and the fifth sensor 124 may be used to control and adjust the pressure based upon the measured inlet 84 and outlet 86 pressures of the first valve 68. As shown in FIG. 4, the third controller 116 may generally control the inlet 84 pressure of the first valve 68. Additionally, although it is not shown in FIG. 4, the console 12 may include the second controller 82 and the third valve 80 which is also in communication with the first pathway 60.

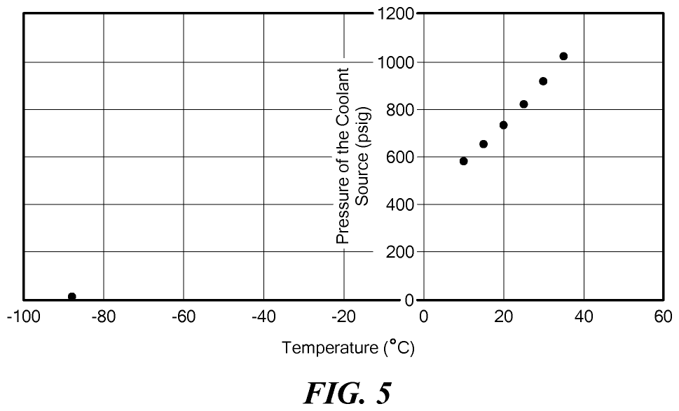

Now referring to FIG. 5, is an example graph showing pressure measurements of coolant that may be used in the console 12 in pounds per square inch gauge (“PSIG”) on the y-axis and temperature in degrees Celsius (“° C.”) on the x-axis. The y-axis demonstrates the pressure of the coolant source 42 within the console 12 and the x-axis is the environmental operating temperature outside the console 12. Generally, the environmental operating temperature outside the console 12 is between approximately 15° C. and 30° C. This is generally the range of temperatures that can be found in the location when the catheter system 10 is in use with the patient. For example, this may be the temperature of a surgical room, procedure room, or another type of area where procedures are performed with the catheter system 10. As can be seen in FIG. 5, the relationship between the pressure of the coolant source 42 and the environmental operating temperature outside the console 12 is linear. As the environmental operating temperature of the console 12 increases, the pressure of the coolant source 42 also increases.

The various components of the console 12 as shown, for example, in FIGS. 2-4, may be used to help control the pressure of the coolant source 42 that is delivered to and removed from the medical device 16. For example, the controllers 70, 82, and 116 within the console 12 may be set to proportionally increase or decrease the pressure in the particular pathway 60, 62, and 63 based upon the temperature measured by the third sensor 112 which may be a temperature sensor. As shown in FIG. 4, the third controller 116 may be used to proportionally increase or decrease the pressure of the coolant source 42 based upon the temperature measured by the temperature sensor 112. The pressure setpoint which is associated with each of the controllers 70, 82, and/or 116 may also be updated between each treatment that is delivered by the medical device 16 or at certain preset time intervals. For example, if the medical device 16 is in a mapping mode, the pressure setpoint of each of or any one of the controllers 70, 82, and/or 116 may be updated between each mapping session. The correlation between the measured environmental operating temperature that is measured using the third sensor 112 and the measured results of any mapping that is taking place when the temperature target is within a particular range can be used to set particular preset parameters to control the pressure in different environmental operating temperatures in one or more than one of the controllers 70, 82, and 116. The third controller 116 may use the correlation between the measured environmental temperature and the measured results of any mapping that is taking place by the medical device 16 and when the temperature target is within a particular range these preset parameters may be used to control the pressure in different environmental operating temperatures.

The pressure of the coolant source 42 may change significantly depending upon the environmental operating temperature. This variability in the pressure of the coolant source 42 can impact the pressure of the first valve 68, specifically at the inlet 84 of the first valve 68. The injection pressure in the first pathway 60, as measured by the first sensor 72, that is needed when cryomapping is occurring, is lower than the injection pressure in the first pathway 60 that is needed for cryoablation procedures. The measurements made by the first sensor 72 are generally the measurements of the pressure from the outlet 86 of the first valve 68. Additionally, the outlet pressure of the first valve 68 used for cryomapping may be lower if the amount of heat load on the medical device 16 is small. For example, when a procedure involves a physically small patient such as a pediatric case the heat load on the medical device 16 may be small and therefore the outlet pressure of the first valve 68 during cryomapping may be set at a lower pressure. The injection pressure, which corresponds to the outlet pressure of the first valve 68 also has a proportional relationship to the environmental operating temperature outside the console 12. Generally, when the environmental operating temperature outside the console 12 is higher a higher injection pressure is also needed for the medical device 16. It can be challenging for the first valve 68 to make small adjustments to the outlet 86 and/or inlet 84 pressure when the difference between the inlet 84 pressure and the outlet 86 pressure of the first valve 68 is high. For example, the pressure that is set during cryo-mapping may be defaulted to room temperature, at approximately 22° C. Alternatively, if the room temperature falls lower than 22° C. from 22° C. to 15° C. there may be a lower preset pressure setpoint for the first pathway 60 during cryo-mapping. If the temperature of the room is above room temperature, for example, from around 22° C. to 30° C., the pressure setpoint for the first pathway 60 may be set at a higher preset pressure setpoint. In pediatric cases where the heat load on the medical device 16 may be small, two additional potential inputs into injection pressure control algorithms may be set based upon a particular patient's age (as indicated by date of birth) as well as weight.

It will be appreciated by persons skilled in the art that the present invention is not limited to what has been particularly shown and described herein above. In addition, unless mention was made above to the contrary, it should be noted that all of the accompanying drawings are not to scale. A variety of modifications and variations are possible in light of the above teachings without departing from the scope and spirit of the invention, which is limited only by the following claims.

We claim:

1. A system for cryoablation, the system having:
a first pathway;
a treatment element, the first pathway being in communication with the treatment element;
a first controller, at least one first valve, and a sensor, the first controller, the at least one first valve, and the sensor all being disposed in the first pathway;
a second controller and a second valve, the second controller and the second valve being in communication with the first pathway; and
a pressurized coolant supply in fluid communication with the first pathway.

2. The system of claim 1, wherein the first controller is a first proportional integral derivative controller and the second controller is a second proportional integral derivative controller.

3. The system of claim 1, wherein the system further comprises an interior portion and an exterior portion, the exterior portion of the system including a temperature sensor configured to record the temperature on the exterior portion of the system and the temperature sensor is in communication with the first controller.

4. The system of claim 3, wherein the exterior portion of the system further includes a humidity sensor configured to record the humidity on the exterior portion of the system and the humidity sensor is in communication with the first controller.

5. The system of claim 4, wherein the first pathway is configured to be activated when the treatment element is in a mapping mode and when the treatment element is in the mapping mode, the first controller sets a mapping pressure based upon the temperature measured by the temperature sensor.

6. The system of claim 5, wherein at least one first valve has an inlet and an outlet, the mapping pressure is determined based upon an inlet pressure from the at least one first valve which is measured by the first sensor.

7. The system of claim 6, wherein the first controller is configured to proportionally decrease the mapping pressure based upon a first preset temperature range that is measured by the temperature sensor.

8. The system of claim 1, wherein the second controller and the second valve are disposed within the first pathway.

9. The system of claim 8, further comprising a second pathway, the second pathway being a vacuum pathway and the first pathway being an injection pathway.

10. The system of claim 9, wherein the second controller and the second valve are disposed within the second pathway.

11. A system for cryoablation, the system having an interior portion, an exterior portion and a treatment element, the system comprising:
a first pathway in the interior portion of the system, the first pathway having a first proportional integral derivative controller, a first valve, and a first pressure transducer;
a second pathway in the interior portion of the system, the second pathway being in communication with a scavenging connector, the second pathway having a second proportional integral derivative controller and a second valve;
a pressurized coolant supply in fluid communication with the first pathway; and
a connector in communication with the treatment element, the connector having a vacuum lumen and an injection lumen.

12. The system of claim 11, where the vacuum lumen and the injection lumen are in fluid communication with the treatment element through the connector.

13. The system of claim 11, further comprising a temperature sensor, the temperature sensor being disposed on the exterior portion of the system and being configured to record the temperature on the exterior portion of the system.

14. The system of claim 13, wherein the first pathway is configured to be activated when the treatment element is in a mapping mode and when the treatment element is in the mapping mode the first proportional integral derivative controller sets a mapping pressure based upon a temperature measured by the temperature sensor.

15. The system of claim 14, wherein the first valve has an inlet and an outlet, the mapping pressure is determined based upon an inlet pressure from the first valve which is measured by the first pressure transducer.

16. The system of claim 15, wherein the first proportional integral derivative controller is configured to proportionally decrease the mapping pressure based upon a first preset temperature range that is measured by the temperature sensor.
